(19) United States
(12) Patent Application Publication
(10) Pub. No.: US 2026/0102053 A1
(43) Pub. Date: Apr. 16, 2026

(54) ENDOSCOPE, ENDOSCOPE CAP, AND ENDOSCOPE SYSTEM

(71) Applicant: OLYMPUS MEDICAL SYSTEMS CORP., Tokyo (JP)

(72) Inventor: Koichi TAKAYAMA, Tokyo (JP)

(73) Assignee: OLYMPUS MEDICAL SYSTEMS CORP., Tokyo (JP)

(21) Appl. No.: 19/413,221

(22) Filed: Dec. 9, 2025

Related U.S. Application Data
(63) Continuation of application No. PCT/JP2023/021963, filed on Jun. 13, 2023.

(51) Int. Cl. A61B 1/00; A61B 1/005; A61B 1/018; A61B 90/00

(52) U.S. Cl. CPC A61B 1/00097; A61B 1/00006; A61B 1/00045; A61B 1/00114; A61B 1/00137; A61B 1/005; A61B 1/018; A61B 90/06; A61B 2090/064; A61B 2560/0462; A61B 2562/0247; A61B 2562/043; A61B 2562/227

(57) ABSTRACT

An endoscope includes pressure sensors including respective pressure-sensitive portions. The endoscope further includes an insertion portion including a distal-end region where the pressure sensors are arranged inside the distal-end region. The distal-end region includes a first region covering the pressure-sensitive portions and having a first rigidity higher than a second rigidity, and a second region including an outer surface of the insertion portion and having the second rigidity.

### CROSS REFERENCE TO RELATED APPLICATION

This application is a continuation of International Application No. PCT/JP2023/021963, filed on Jun. 13, 2023, the entire contents of which are incorporated herein by reference.

### FIELD

The present disclosure relates to an endoscope, an endoscope cap, and an endoscope system.

### BACKGROUND

Conventionally, endoscopes have been widely used, for example, in medical fields, industrial fields, and the like. Medical endoscopes that are used in the medical fields have a function of acquiring observation target images including a lesion part, etc., inside a body cavity, an organ, or the like of a subject such as a living body, by an insertion portion provided with an image pickup unit being inserted into the body cavity, the organ, or the like. Furthermore, the images thus acquired are used for an image diagnosis or the like for an observation or an examination of a lesion part and the like.

An endoscopy that is performed by using a conventional medical endoscope includes, for example, an upper gastrointestinal endoscopy, a colonoscopy, etc. The upper gastrointestinal endoscopy is performed mainly for observation, examination, and treatment of an inside of upper gastrointestinal tracts including an esophagus, a stomach, a duodenum, and the like. The colonoscopy is performed mainly for observation, examination, and treatment of an inside of a lumen of a large intestine. Such endoscopies are widely performed.

In general, in an endoscopy, an insertion operation is performed in which a distal-end constituting portion of an insertion portion, which has been inserted from an oral cavity, an anus, or the like into a lumen, is advanced into a deep part along the lumen. This insertion operation includes predetermined operations such as a predetermined bending operation that is performed as needed to actively bend a bending portion of the insertion portion, or a twisting operation applied to the insertion portion. During execution of such an insertion operation, endoscopic image data is continuously acquired by an image pickup unit provided at the distal-end constituting portion of the insertion portion, and predetermined image processing is performed. Then, images generated based on the endoscopic image data are displayed consecutively on a display apparatus. This enables a user such as an operator (hereinafter, referred to as an operator or the like) to perform real-time observation of endoscopic images.

The insertion operation of the insertion portion at this time is sometimes performed, for example, with the distal-end constituting portion of the insertion portion being pressed against an inner wall portion, etc., of a lumen. In this case, it has been normal for an operator or the like to appropriately adjust a load applied to the inner wall portion, etc., that is, an amount of pressing force or an amount of load force (hereinafter, simply referred to as a force) when the distal-end constituting portion is pressed against the inner wall portion, etc., while confirming an endoscopic image and based on feeling of hand and fingers.

On the other hand, an organ and the like, which are targets of observation and examination in an endoscopy, are arranged complicatedly in a subject such as a living body, and include a lot of flex portions. Furthermore, it is known that the organ and the like are not always maintained in certain shapes in a subject, and the shapes are constantly changing according to a posture of the subject, for example.

Accordingly, since an insertion operation in an endoscopy is performed by inserting an insertion portion into an organ and the like whose arrangement is complicated and direct visual recognition thereof is impossible, the insertion operation tends to be affected by the skill of an operator or the like. For example, during the insertion operation, if the distal-end constituting portion is pressed against the inner wall portion of the organ and the like with an excessive force, a burden is imposed on the subject (patient, etc.) and the organ and the like are likely to be affected.

Thus, in an endoscopy, a stable insertion operation is always required to be carried out while always ensuring a stable amount of pressing force, regardless of the skill of the operator or the like.

In view of the above, for example, Japanese Patent Application Laid-Open Publication No. 2016-152863, International Patent Publication No. WO 2021/176530, etc., provide various proposals for an endoscope having a function for detecting an amount of force applied to a distal-end constituting portion of an insertion portion.

The technology disclosed in the above-described Japanese Patent Application Laid-Open Publication No. 2016-152863 includes a configuration in which a pressure sensor, which is a circular-ring-shaped hood-cap type load detection apparatus, is attached to an outer lateral side of the distal-end constituting portion of the insertion portion of the endoscope.

The technology disclosed in the above-described International Patent Publication No. WO 2021/176530 relates to an endoscope hood that is attached to the distal-end constituting portion of the insertion portion of the endoscope, and the endoscope hood includes a main body portion that is constituted of a hood portion arranged on a distal end side relative to the insertion portion and an attaching portion fitted on the outer circumference of the insertion portion, and a pressure sensor provided at the attaching portion.

### SUMMARY

According to aspects of the present disclosure, an endoscope is provided, which includes pressure sensors and an insertion portion. The pressure sensors include respective pressure-sensitive portions. The insertion portion includes a distal-end region where the pressure sensors are arranged inside the distal-end region. The distal-end region includes a first region covering the pressure-sensitive portions and having a first rigidity higher than a second rigidity. The distal-end region further includes a second region including an outer surface of the insertion portion and having the second rigidity.

According to aspects of the present disclosure, further provided is an endoscope cap configured to be detachably attached to a distal end of an insertion portion of an endoscope. The endoscope cap includes pressure sensors disposed inside the endoscope cap and including respective pressure-sensitive portions. The endoscope cap further includes a first region covering the pressure-sensitive portions and having a first rigidity higher than a second rigidity. The endoscope cap further includes a second region including an outer surface of the endoscope cap and having the second rigidity.

According to aspects of the present disclosure, further provided is an endoscope system that includes an endoscope and a processor. The endoscope includes pressure sensors and an insertion portion. The pressure sensors include respective pressure-sensitive portions. The insertion portion includes a distal-end region where the pressure sensors are arranged inside the distal-end region. The distal-end region includes a first region covering the pressure-sensitive portions and having a first rigidity higher than a second rigidity. The distal-end region further includes a second region including an outer surface of the insertion portion and having the second rigidity. The processor is configured to automatically control at least one of an insertion direction or an insertion force of the insertion portion based on output signals from the pressure sensors.

### BRIEF DESCRIPTION OF THE DRAWINGS

FIG. 1 is a schematic configuration view showing an overall configuration of an endoscope system including an endoscope in a first embodiment of the present disclosure.

FIG. 2 is an appearance perspective view showing, in an enlarged manner, a distal end part of an insertion portion in the endoscope in the first embodiment of the present disclosure.

FIG. 3 is a longitudinal sectional view along an imaginary plane indicated by an arrow [3] in FIG. 2.

FIG. 4 is a front view viewed in a direction indicated by an arrow [4] in FIG. 2.

FIG. 5 is a cross-sectional view along a line indicated by an arrow [5] in FIG. 3.

FIG. 6 is a cross-sectional view along a line indicated by an arrow [6] in FIG. 3.

FIG. 7 is a cross-sectional view along a line indicated by an arrow [7] in FIG. 3.

FIG. 8 is a cross-sectional view along a line indicated by an arrow [8] in FIG. 3.

FIG. 9 is a conceptual view showing operation of the endoscope in the first embodiment of the present disclosure, which shows a state where the insertion portion is inserted into a subject.

FIG. 10 is a conceptual view showing, in an exploded manner, a cross section along a curved line indicated by a reference sign [10] in FIG. 4.

FIG. 11 is a conceptual view of a display screen displayed on a monitor while an examination such as a colonoscopy is performed by using the endoscope system in FIG. 1.

FIG. 12 is an exploded perspective view showing, in an enlarged manner, an endoscope cap in a second embodiment of the present disclosure, and a distal end part of an insertion portion to which the endoscope cap is mounted.

FIG. 13 is an appearance perspective view showing a state where the endoscope cap in FIG. 12 is mounted to the distal end part of the insertion portion.

FIG. 14 is a longitudinal sectional view along an imaginary plane indicated by the arrow [14] in FIG. 13.

FIG. 15 is a cross-sectional view along a line indicated by an arrow [15] in FIG. 14.

FIG. 16 is a cross-sectional view along a line indicated by an arrow [16] in FIG. 14.

FIG. 17 is a cross-sectional view along a line indicated by an arrow [17] in FIG. 14.

FIG. 18 is a cross-sectional view along a line indicated by an arrow [18] in FIG. 14.

FIG. 19 is a cross-sectional view along a line indicated by an arrow [19] in FIG. 14.

### DESCRIPTION OF EMBODIMENTS

Hereinafter, the present disclosure will be described by illustrated embodiments. The respective drawings used in the following description are schematically shown, and in order to show each of the constituent elements in a size recognizable in the drawings, there is a case where the dimensional relationship, scales, etc., of the respective members are made different for each of the constituent elements. Therefore, the present disclosure is not limited only to the illustrated forms with regard to the number, the shapes, the ratio of the sizes, the relative positional relationship, etc., of the respective constituent elements illustrated in each of the drawings.

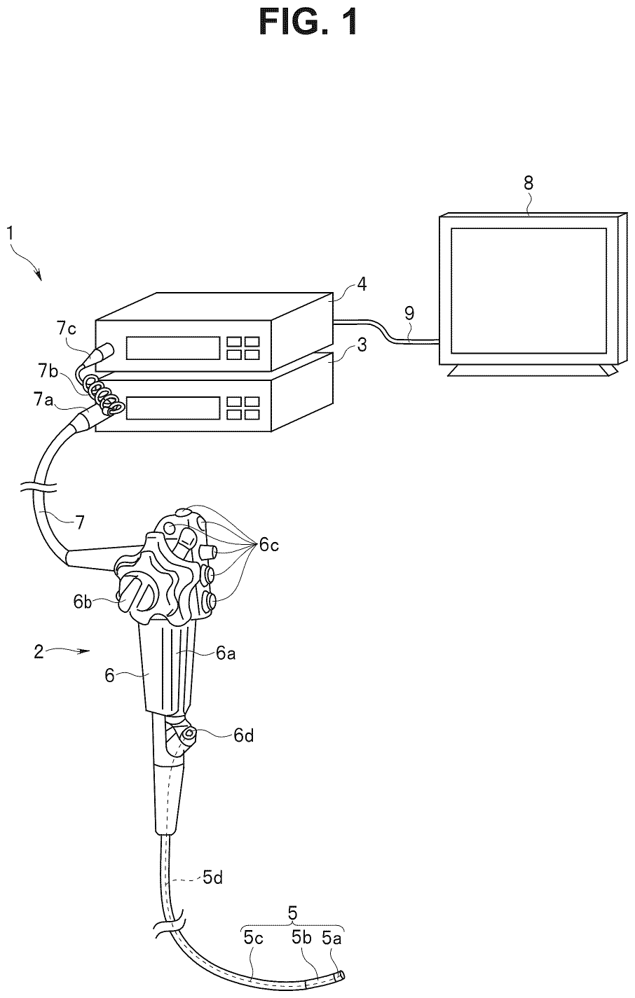

First, before describing a detailed configuration of an endoscope in a first embodiment of the present disclosure, a schematic configuration of an entire endoscope system including the endoscope will be described with reference to FIG. 1. FIG. 1 is a schematic configuration view showing an overall configuration of the endoscope system including the endoscope in the first embodiment of the present disclosure.

As shown in FIG. 1, an endoscope system 1 includes an endoscope 2, a light source apparatus 3, a processor 4, a monitor 8, and the like. Note that the endoscope system 1 exemplifies a colonoscope system for performing a colonoscopy for observing an inside of a large intestine of a subject (patient, etc.) as an observation target. A basic configuration of the endoscope system 1 shown in FIG. 1 is substantially the same as that of a conventional endoscope system of the same type. Further, note that the light source apparatus 3, the processor 4, and the monitor 8 may be integrated with the endoscope 2. In this case, the endoscope according to aspects of the present disclosure may be configured as an endoscope system.

The endoscope 2 includes an insertion portion 5, an operation portion 6, a universal cord 7, and the like.

The insertion portion 5 is a constituent member configured to be inserted into a subject such as a living body. The insertion portion 5 is formed by a distal-end constituting portion 5a, a bending portion 5b, and a flexible tube portion 5c being consecutively connected in this order from the distal end side. The insertion portion 5 is formed in substantially an elongated tube shape as a whole. The insertion portion 5 includes, inside thereof, a treatment instrument insertion channel 5d as a conduit through which a treatment instrument for endoscope, not shown, is inserted. The treatment instrument insertion channel 5d is provided so as to pass through from the distal end to the proximal end of the insertion portion 5. Furthermore, the operation portion 6 is connected to the proximal end side of the insertion portion 5.

The distal-end constituting portion 5a includes, inside thereof, various constituent members such as an image pickup unit and an illumination unit (not shown in FIG. 1). Here, the image pickup unit is an electronic device unit constituted of a photoelectric conversion device, an optical lens (image pickup lens), and the like that are configured to acquire image information (still image and moving image) of an observation target (for example, an inner wall surface, etc., of an organ such as a large intestine) in a subject. The illumination unit is a constituent unit including an optical element (illumination lens) configured to emit a light flux guided from the light source apparatus 3 forward from a distal end surface of the distal-end constituting portion 5a, to illuminate an observation target region including a lesion part and the like in the subject. Note that a detailed inner configuration of the distal-end constituting portion 5a of the insertion portion 5 in the endoscope 2 in the present embodiment will be described later.

The operation portion 6 is connected to the proximal end of the insertion portion 5. The operation portion 6 includes an operation portion main body 6a, a bending operation knob 6b, a plurality of operation members 6c, a treatment instrument insertion port 6d, and the like.

The operation portion main body 6a is formed in substantially a box shape as a whole, and configures a grasping portion configured to be grasped by an operator, or the like, as a user of the endoscope 2. As described above, the insertion portion 5 extends from the operation portion main body 6a.

The bending operation knob 6b and the plurality of operation members 6c are operation members for performing various kinds of operations of the endoscope 2. The bending operation knob 6b and the plurality of operation members 6c are provided respectively at predetermined positions on an outer surface of the operation portion main body 6a.

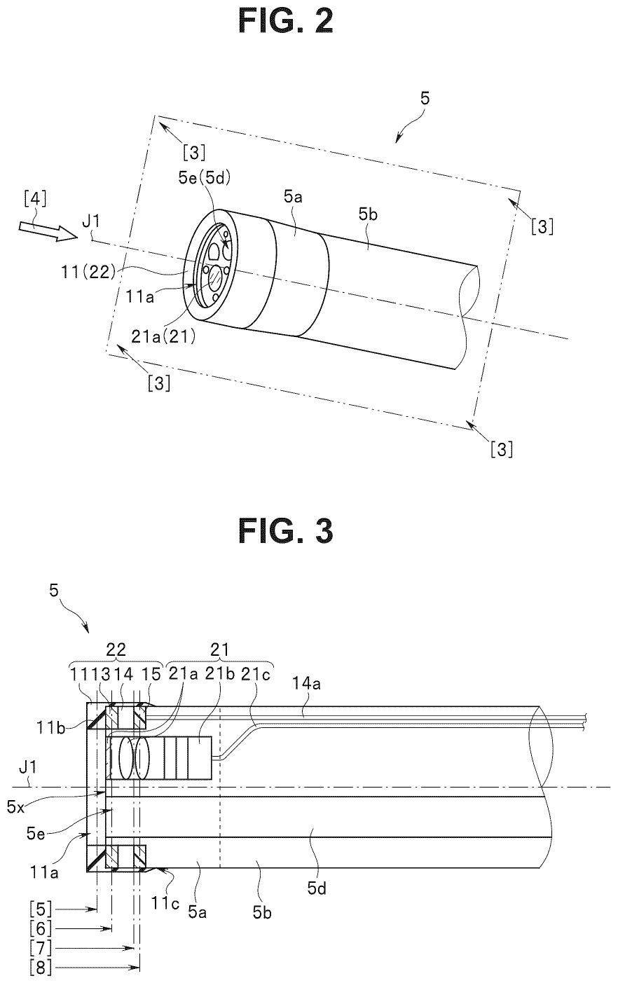

The treatment instrument insertion port 6d is provided at a predetermined position closer to the distal end of the operation portion main body 6a. The treatment instrument insertion port 6d is a proximal-end-side opening of the treatment instrument insertion channel 5d of the insertion portion 5. The treatment instrument insertion channel 5d is connected, at the distal end side thereof, to a channel opening 5e (not shown in FIG. 1, see FIG. 2, etc., to be described later) which is a distal-end opening of the distal-end constituting portion 5a.

Such a configuration enables the treatment instrument for endoscope (not shown) inserted from the treatment instrument insertion port 6d to pass through the treatment instrument insertion channel 5d, and then protrude outward from the channel opening 5e of the distal-end constituting portion 5a.

The universal cord 7 is a connection cord for connecting the endoscope 2 to the light source apparatus 3 and the processor 4. To this end, the universal cord 7 is formed of a tubular member extended from a lateral side of the operation portion main body 6a of the operation portion 6. The universal cord 7 includes, at the distal end thereof, a scope connector 7a. The scope connector 7a is connected to a front surface panel of the light source apparatus 3.

An electric cable 7b extends from the scope connector 7a. A connector 7c is provided at the distal end of the electric cable 7b. The connector 7c is connected to a front surface panel of the processor 4. Inside the universal cord 7, various signal transmission cables, an optical fiber cable, and the like (not shown) are inserted.

The light source apparatus 3 is configured to supply illumination light to the illumination unit provided in the distal-end constituting portion 5a of the insertion portion 5 of the endoscope 2. The illumination light emitted from the light source apparatus 3 is transmitted to the illumination unit of the distal-end constituting portion 5a through the optical fiber cable, etc., (not shown) disposed by passing through from the scope connector 7a to the universal cord 7, the operation portion 6, and the insertion portion 5. Then, the illumination light passes through the illumination lens, etc., included in the illumination unit of the distal-end constituting portion 5a, to be applied toward the observation target region in front of the distal-end constituting portion 5a.

The processor 4 is a control apparatus and also a signal processing apparatus that include a control circuit configured to control the entire endoscope system 1, a signal processing circuit, and the like. The control circuit in the processor 4 receives, for example, an operation instruction signal from the operation member 6c of the operation portion 6 of the endoscope 2, and outputs, for example, various control signals for performing driving control of the image pickup unit, the light source apparatus, the illumination unit, and the like. In addition, the signal processing circuit receives, for example, an image pickup signal from the image pickup unit provided in the distal-end constituting portion 5a of the insertion portion 5 of the endoscope 2, to perform predetermined image signal processing.

For this purpose, the processor 4 and the image pickup unit are electrically connected to each other by a signal transmission cable (not shown). The signal transmission cable is disposed so as to pass through from the connector 7c to the image pickup unit of the distal-end constituting portion 5a, via the electric cable 7b, the scope connector 7a, the universal cord 7, the operation portion 6, and the insertion portion 5.

With such a configuration, the control signal output from the processor 4, the image pickup signal output from the image pickup unit, and the like are transmitted between the image pickup unit and the processor 4 through the signal transmission cable. Note that one form of the signal transmission cable includes, for example, a composite cable configured by a bundle of a plurality of cables covered with an outer shield, an outer tube, etc.

The monitor 8 is a display apparatus configured to receive an image signal, etc., output from the processor 4, and display an endoscopic image, various kinds of information, and the like, in predetermined forms. To this end, the monitor 8 and the processor 4 are electrically connected to each other using a video cable 9 in a predetermined form. Note that a form of the monitor 8 includes a display device configured by using, for example, a general liquid crystal panel or the like.

The light source apparatus 3 and the processor 4 are not limited to be configured separately from each other as shown in the configuration example shown in FIG. 1. For example, the light source apparatus 3 and the processor 4 may be configured integrally.

In addition, the configuration example of the illumination unit is not limited to the above-described configuration example (form in which the illumination light from the light source apparatus is transmitted to the distal-end constituting portion through the optical fiber cable, etc.). As another form of the illumination unit other than the above configuration example, for example, a light-emitting element such as a light emitting diode (LED) as an illumination light source is provided in the distal-end constituting portion, and power supply to the illumination light source (LED) and light emission control of the LED can be controlled by the control circuit of the processor 4.

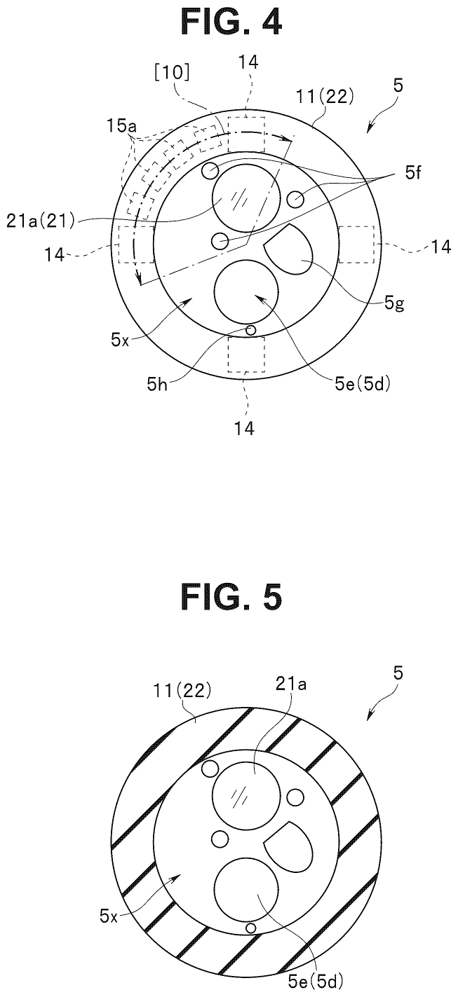

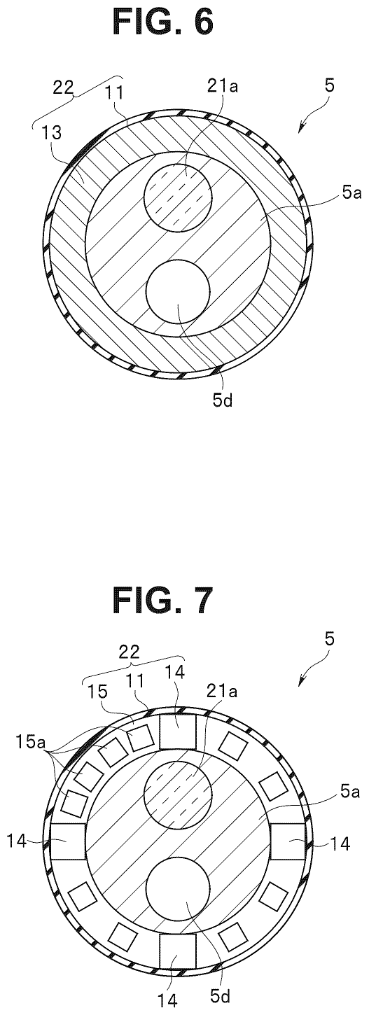

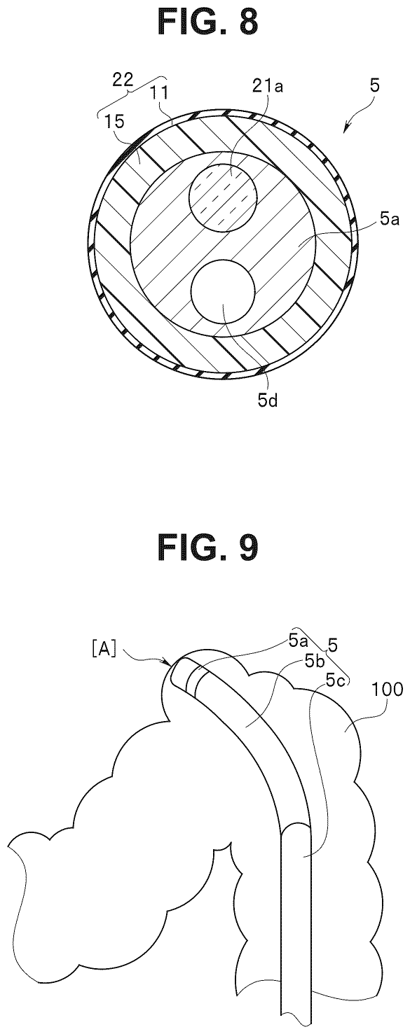

Next, the detailed configuration of the distal-end constituting portion 5a of the insertion portion 5 in the endoscope 2 in the present embodiment will be described below with reference to FIGS. 2 to 8. FIGS. 2 to 8 each show, in an enlarged manner, at least a part of the distal end part of the insertion portion in the endoscope in the present embodiment. Among these figures, FIG. 2 is the appearance perspective view of the distal end part of the insertion portion. FIG. 3 is a longitudinal sectional view along the imaginary plane indicated by the arrow [3] in FIG. 2. FIG. 4 is a front view viewed in a direction indicated by the arrow [4] in FIG. 2. FIG. 5 is a cross-sectional view along the line indicated by the arrow [5] in FIG. 3. FIG. 6 is a cross-sectional view along the line indicated by the arrow [6] in FIG. 3. FIG. 7 is a cross-sectional view along the line indicated by the arrow [7] in FIG. 3. FIG. 8 is a cross-sectional view along the line indicated by the arrow [8] in FIG. 3.

Note that, in each of the cross-sectional views in FIGS. 6 to 8, only the constituent members included mainly in a load detection unit (detailed later) are shown, and illustrations of other constituent members are partly omitted to avoid complication of the drawings.

The distal-end constituting portion 5a of the insertion portion 5 in the endoscope 2 in the present embodiment includes, as shown in FIG. 2, FIG. 3, etc., an image pickup unit 21, the illumination unit (not shown), the treatment instrument insertion channel 5d (a part thereof), a load detection unit 22, etc. (see FIG. 3).

The image pickup unit 21 is a constituent unit which includes an image pickup lens 21a, an image pickup device 21b, a signal transmission cable 21c, and the like and configured to acquire a desired image. The illumination unit is a constituent unit which includes an illumination lens 5f as shown in FIG. 4, and the like, and is configured to illuminate a target. The treatment instrument insertion channel 5d is a hollow tubular member that includes, at the distal end thereof, the channel opening 5e, and passes through the inside of the insertion portion 5, to reach the inside of the operation portion 6.

Note that the image pickup unit 21, the illumination unit, and the treatment instrument insertion channel 5d have configurations substantially the same as those applied to conventional endoscopes of the same type. Accordingly, the description of the detailed configurations of these will be omitted.

For example, on a front surface 5x of the distal-end constituting portion 5a, various constituent members are disposed, as shown in FIG. 4, etc. The constituent members include the image pickup lens 21a as a part of the image pickup unit 21, the illumination lens 5f as a part of the illumination unit, a gas/liquid feeding nozzle 5g, an auxiliary liquid feeding port 5h, the channel opening 5e, and the like.

The load detection unit 22 is a constituent unit disposed at the distal end part of the distal-end constituting portion 5a and configured to detect an amount of force (load) applied to the distal end part of the insertion portion 5. As shown in FIG. 2, FIG. 3, etc., the load detection unit 22 includes a distal end cap 11, a rigid ring 13, a plurality of pressure sensors 14, a sensor substrate 15, and the like.

The distal end cap 11 is an exterior member configured to cover mainly the lateral surface on a side closer to the distal end and a part (outer peripheral edge portion) of the front surface, of an outside surface of the distal-end constituting portion 5a, and forms a part of the outer surface of the insertion portion 5. The distal end cap 11 is formed in substantially a cylindrical shape as a whole. The distal end cap 11 includes, at one end portion (distal end side) thereof, an inward flange 11b formed so as to have a first opening 11a. The distal end cap 11 includes, at another end portion (proximal end side) thereof, a second opening 11c. The first opening 11a and the second opening 11c are connected to each other by a through hole penetrating through the distal end cap 11.

Here, the first opening 11a has a size (area) that allows exposure, without shielding, of all the front surfaces of the various constituent members (the image pickup lens 21a, the illumination lens 5f, the gas/liquid feeding nozzle 5g, the auxiliary liquid feeding port 5h, the channel opening 5e, etc.) which are disposed on the front surface 5x of the distal-end constituting portion 5a. Note that the distal end cap 11 is formed of a flexible material having low rigidity (for example, rubber material).

The distal end cap 11 includes, in the inner region thereof, the rigid ring 13, the pressure sensors 14, the sensor substrate 15 that are disposed so as to align in this order from the distal end side toward the proximal end side. These constituent members (the rigid ring 13, the pressure sensors 14, and the sensor substrate 15) are arranged at a part closer to the distal end inside the distal-end constituting portion 5a of the insertion portion 5.

The rigid ring 13 is a member formed in substantially a circular ring shape as a whole. The rigid ring 13 is disposed such that the outer circumferential surface thereof is along the inner wall surface of the lateral surface of the distal end cap 11. In addition, the rigid ring 13 is disposed such that the front end surface thereof is in contact with the inner surface of the inward flange 11b of the distal end cap 11. In this case, the front end surface of the rigid ring 13 is fixed, by an adhesive, for example, integrally with the inner surface of the inward flange 11b. Thus, the rigid ring 13 is arranged at the part which is closer to the distal end and in the vicinity of the outer peripheral edge portion of the distal-end constituting portion 5a.

The rigid ring 13 is formed of a hard material having high rigidity (for example, a metal material such as stainless steel (e.g., SUS), a hard resin, etc.). In other words, the rigid ring 13 may include at least one of a metal or a hard resin. Accordingly, the rigid ring 13 is configured so as to have the rigidity higher than the rigidity of the distal end cap 11. Note that the rigid ring 13 has a thickness dimension of approximately 0.005 mm to 3 mm, and a diameter dimension of approximately 5 mm to 20 mm.

Each of the pressure sensors 14 is a sensor device arranged at the part closer to the distal end inside the insertion portion 5 (inside the distal end cap 11) and configured to detect a pressure applied to the distal-end constituting portion 5a. The plurality of pressure sensors 14 are mounted on a mounting surface of the sensor substrate 15. A pressure-sensitive portion (front surface) of each of the pressure sensors 14 is arranged so as to face the rear end surface of the rigid ring 13. At this time, the pressure-sensitive portion of each of the pressure sensors 14 and the rear end surface of the rigid ring 13 are arranged facing each other, with almost no gap therebetween (that is, substantially a contact state) or with a slight gap (for example, approximately 0.1 mm) therebetween.

As described above, the front end surface of the rigid ring 13 is fixed integrally with the inner surface of the inward flange 11b of the distal end cap 11. Furthermore, the rear end surface of the rigid ring 13 is arranged, in a contact manner, at a position opposed to the pressure-sensitive portions of the pressure sensors 14. Here, the surface with which the pressure-sensitive portions of the pressure sensors 14 are in contact is referred to as an inner contact surface.

In addition, in the distal end cap 11, a predetermined region including the rigid ring 13 is referred to as a first region. Furthermore, in the distal end cap 11, a predetermined region including the outer surface is referred to as a second region. Hereinafter, a region including the pressure sensors 14, the first region, and the second region may be referred to as a “distal-end region,” which is a positional concept indicating a region closer to the distal end of the distal-end constituting portion 5a of the insertion portion 5.

In this case, the distal end cap 11 of the insertion portion 5 is configured such that the rigidity of the first region including the inner contact surface (rigid ring 13) with which the respective pressure-sensitive portions of the pressure sensors 14 are in contact is higher than the rigidity of the second region including the outer surface of the distal end cap 11 of the insertion portion 5. In other words, the rigid ring 13 which is a contact member has the rigidity higher than that of the second region.

In the configuration example of the present embodiment, the pressure-sensitive portions of the pressure sensors 14 are in contact with the rear end surface of the rigid ring 13. Thus, in the configuration example, the inner contact surface is the rear end surface of the rigid ring 13. In this case, the rigid ring 13 is the contact member provided at a position with which the pressure-sensitive portions of the pressure sensors 14 are in contact. In detail, the rigid ring 13 as the contact member is arranged between the distal end cap 11 and the pressure-sensitive portions. With such a configuration, a force applied to the outer surface of the insertion portion 5 (distal end cap 11) is transmitted to the pressure-sensitive portions of the pressure sensors 14 through the rigid ring 13 as the contact member.

The plurality of pressure sensors 14 are arranged at substantially equal intervals along the circumferential direction around a center axis J1 of the distal end cap 11. At least two pressure sensors 14 may be provided.

In the present embodiment, the configuration example is shown in which the four pressure sensors 14 are arranged at substantially equal intervals along the circumferential direction around the center axis J1 of the distal end cap 11. Note that, for example, piezoelectric elements are used as the pressure sensors 14.

The sensor substrate 15 has substantially a circular ring shape, and is disposed such that the outer circumference thereof is along the inner surface of the lateral surface of the distal end cap 11. The pressure sensors 14 are mounted on the mounting surface (front surface side) of the sensor substrate 15. In addition, on the same mounting surface of the sensor substrate 15, an electronic circuit (for example, an analog-to-digital (AD) conversion circuit) that receives output signals of the pressure sensors 14 to perform predetermined signal processing on the received signals is mounted.

Note that, in order to avoid complication of the drawings, illustration of the electronic circuit such as the AD conversion circuit is partly omitted. Although detailed description will be made later, FIG. 4, FIG. 7, etc., partly show, in a conceptual manner, the state where a plurality of electronic components 15a as the members constituting the AD conversion circuit, etc., are mounted on the mounting surface of the sensor substrate 15.

As shown in FIG. 3, a signal cable 14a extends from the pressure sensors 14 (the sensor substrate 15 on which the pressure sensors 14 are mounted) toward the proximal end side of the insertion portion 5. The signal cable 14a passes, together with the other signal transmission cable 21c, etc., through the inside of the insertion portion 5. In addition, although illustration is omitted, the signal cable 14a passes through the insertion portion 5, the operation portion 6, the universal cord 7, the scope connector 7a, the electric cable 7b, and the connector 7c, to be connected to the processor 4. Such a configuration allows the output signals of the pressure sensors 14 to be transmitted to the processor 4.

In the endoscope 2 in the present embodiment thus configured, the distal-end constituting portion 5a includes the load detection unit 22. With such a configuration, the endoscope 2 functions as a detection device that detects the amount of force (load) applied mainly to the front surface 5x of the insertion portion 5.

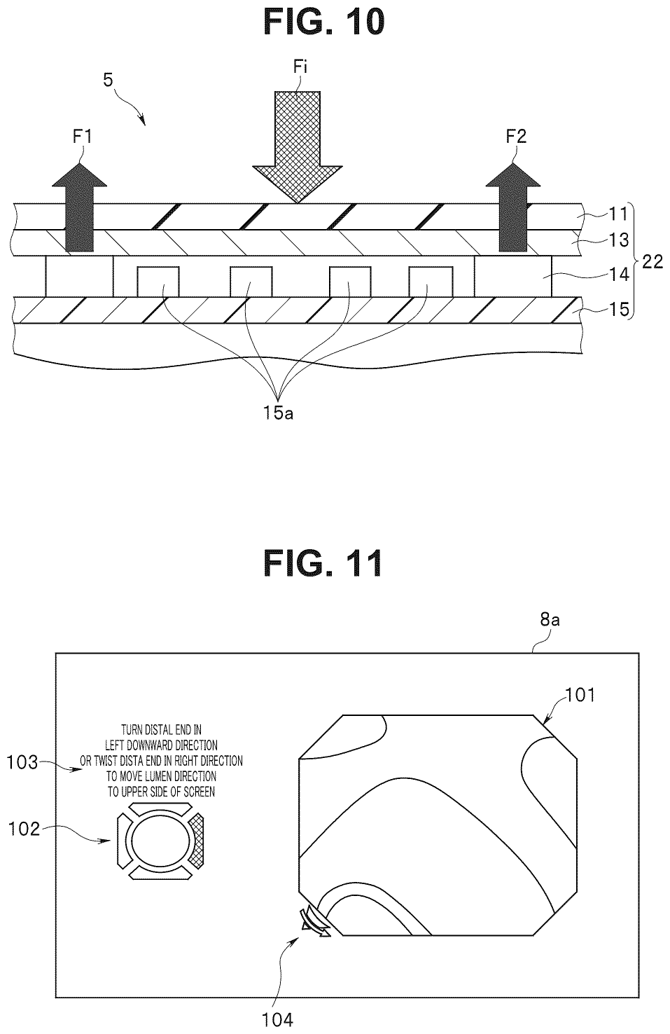

Next, among the operation of the endoscope 2 in the present embodiment, the operation of the load detection unit 22 will be described below with reference to FIGS. 9 and 10. The description below is made assuming the operation in the case where the endoscope system 1 shown in FIG. 1 is used to perform a colonoscopy, for example.

First, the insertion portion 5 of the endoscope 2 in the endoscope system 1 shown in FIG. 1 is inserted into a subject such as a patient as an examination target according to a normal examination procedure (not shown).

Here, FIG. 9 is a conceptual view showing the operation of the endoscope 2 in the first embodiment of the present disclosure, which shows a state where the insertion portion 5 is inserted into the subject.

As shown in FIG. 9, while the colonoscopy is performed, the insertion portion 5 of the endoscope 2 is being inserted into an inner portion of a lumen of an organ 100 such as a large intestine, for example. At this time, the operator, or the like performs an operation for advancing the insertion portion 5 along the inner portion of the lumen in the organ of the subject, while performing a predetermined insertion operation using the bending operation knob 6b and the like of the operation portion 6.

When the insertion portion 5 is inserted along the inner portion of the lumen in the organ, for example, the distal end part of the insertion portion 5 sometimes contacts the inner wall surface or the like of the organ of the subject, as shown in FIG. 9. In this case, if the insertion operation of the insertion portion 5 is continued, the distal end part of the insertion portion 5 is pressed against the inner wall surface of the organ with a strong force. The state shown in FIG. 9 shows that the distal end part of the insertion portion 5 contacts a part of the inner wall surface of the organ of the subject (part indicated by the reference sign [A] in FIG. 9).

When the distal end part of the insertion portion 5 is brought into such a state, a predetermined force is applied to the distal end part of the insertion portion 5 of the endoscope 2. Here, FIG. 10 is a conceptual view showing, in an exploded manner, a cross section along a curved line indicated by the reference sign [10] in FIG. 4. In FIG. 10, the force applied to the distal end part of the insertion portion 5 is indicated by the arrow reference sign Fi. The force Fi is first applied to the outer surface of the distal end cap 11. Then, the inward flange 11b is pressed with the force Fi. The distal end cap 11 (the inward flange 11b) is formed of the flexible material having the low rigidity. Therefore, even if the outer surface of the distal end part of the insertion portion 5 contacts, to press the inner wall surface or the like of the organ 100, there is little influence on the inner wall surface or the like.

When the inward flange 11b is further pressed with the force Fi, the rigid ring 13 is then pressed with the force Fi. The rigid ring 13 is formed of the hard material having the rigidity higher than that of the distal end cap 11. Therefore, when the force Fi is transmitted to the rigid ring 13, the rigid ring 13, as a whole, is pushed in the same direction (the arrow Fi direction in FIG. 10) as the direction in which the force Fi is applied. As a result, the rigid ring 13 presses the pressure-sensitive portions of the pressure sensors 14.

In this case, when the force Fi applied to the outer surface of the distal end cap 11 acts, for example, on the position corresponding to the position where any of the pressure sensors 14 is arranged, the force Fi presses the pressure-sensitive portion of the pressure sensor 14, which corresponds directly to the position on which the force Fi acts, through the rigid ring 13.

On the other hand, in a case where the force Fi applied to the outer surface of the distal end cap 11 acts, for example, on any position in a middle region between the positions where two pressure sensors 14 are respectively arranged, the force Fi presses the respective pressure-sensitive portions of the two pressure sensors 14 such that predetermined forces are respectively applied thereto, through the rigid ring 13.

As shown in FIG. 10, for example, when the force Fi acts on substantially a middle position of the two pressure sensors 14, the rigid ring 13, as a whole, is pushed in the direction of the force Fi without deformation. At this time, reaction forces F1 and F2 are generated at the respective pressure-sensitive portions of the two pressure sensors 14. As a result, the relation of Fi=F+F2 is established, and the pressure sensors 14 can always detect the force Fi applied to a predetermined position of the distal end part of the distal end cap 11 with high accuracy.

At the same time, based on respective output signals of the two pressure sensors 14, the position where the force acts, which is around the center axis J1 of the insertion portion 5, can also be detected.

For example, description will be made assuming a case where, in the middle region between the two pressure sensors 14, the position where the force Fi acts is closer to one of the two pressure sensors 14 (it is assumed that the sensor denoted by the reference sign of the reaction force F1).

In this case, the reaction forces F1 and F2 of the respective pressure sensors 14 satisfy the relation of F1>F2, for example. Also in this case, the relation of Fi=F1+F2 is established. Accordingly, detection of each of the reaction forces F1 and F2 enables the force acting position in the middle region of the two pressure sensors 14 to be specified.

Then, the output signals of the respective pressure sensors 14 are transmitted to the processor 4 through the signal cable 14a. The processor 4 performs predetermined signal processing based on the received output signals of the pressure sensors 14. Then, the processor 4 generates predetermined display information. The generated display information is output to the monitor 8. In response, the monitor 8 displays various kinds of information generated based on the output signals of the pressure sensors 14.

Here, FIG. 11 is a conceptual view of a display screen displayed on the monitor 8 while an examination such as a colonoscopy is performed by using the endoscope system 1 in FIG. 1.

As shown in FIG. 11, a display screen 8a of the monitor 8 shows display examples of a real-time endoscopic image 101, which is in the process of being acquired by the endoscope 2, and various kinds of information generated based on the output signals of the respective pressure sensors 14.

The display examples of the information shown in FIG. 11 include, for example, a distribution of the force applied to the distal end part of the insertion portion 5 (see the reference sign 102 in FIG. 11), a guide for insertion operation of the insertion portion 5 (see the reference sign 103 in FIG. 11), an icon indicating the guide for the insertion operation of the insertion portion 5 (see the reference sign 104 in FIG. 11), etc.

A force distribution display 102 shows the distribution of the force applied to the distal end surface when viewed facing the distal end part of the insertion portion 5, in a form of an icon. In the display example shown in FIG. 11, four regions are shown around the distal end surface of the insertion portion 5. Then, among the four regions, a region including a position to which the force is being applied is indicated by a predetermined colored display (In FIG. 11, the colored display is indicated with the cross-hatching).

Note that, as described above, the present embodiment shows the configuration example in which the four pressure sensors 14 are provided in the distal-end constituting portion 5a of the insertion portion 5. For this reason, the force distribution display 102 in FIG. 11 shows the four regions corresponding respectively to the four pressure sensors 14.

When a predetermined force is applied to the distal end surface of the insertion portion 5 and the force acting position is located in the middle region between the respective pressure sensors 14, the pressure-sensitive portions of at least two pressure sensors 14 in the vicinity of the force acting position reacts.

In such a case, for example, the predetermined colored display is applied to the plurality of display regions corresponding respectively to the two pressure sensors 14 in the vicinity of the force acting position, to thereby enable the display of the force acting position (direction of the force) around the center axis of the insertion portion 5. Furthermore, at this time, for example, the colored display for the plurality of display regions is performed by contrivances such as displaying with gradations of color according to the distance from the force acting position of the pressure sensor 14, to thereby enable the clarification of the force acting position.

As described above, according to the above-described first embodiment, the outer surface of the insertion portion 5 is configured by the distal end cap 11 formed of the flexible material having the low rigidity, and the part with which the pressure-sensitive portions of the pressure sensors 14 are in contact is configured by including the rigid ring 13 formed of the hard material having the high rigidity.

Therefore, at the time of the insertion operation of the insertion portion 5 during the execution of a colonoscopy, for example, even if the distal end part of the insertion portion 5 contacts to press the wall surface and the like of an organ such as a large intestine, there is little influence on the wall surface and the like of the organ. Furthermore, at this time, when a force is applied to the distal end part of the distal end cap 11, the pressure-sensitive portions of the pressure sensors 14 contact the rigid ring 13 formed of the hard material having the high rigidity. This enables the force applied to the distal end part of the insertion portion 5 to be always detected with high accuracy.

In addition, in the inner region of the distal end cap 11, the rigid ring 13 is provided between the rear end surface of the inward flange 11b of the distal end cap 11 and the pressure-sensitive portions of the pressure sensors 14, and a predetermined gap is provided between the inner contact surface of the rigid ring 13 and the plurality of electronic components 15a.

To be more detailed, the rigid ring 13 (contact member) is arranged across the respective pressure-sensitive portions of the at least two pressure sensors 14. In this case, a predetermined gap is provided between a reference surface coplanar with the pressure-sensitive portions and the mounting surface of the sensor substrate 15 on which the pressure sensors 14 are mounted. In addition, the plurality of electronic components 15a are provided within a range of the predetermined gap.

With such a configuration, the force Fi applied to the outer surface of the distal end cap 11 is received by the rigid ring 13. In this configuration, the force Fi does not affect the plurality of electronic components 15a. Therefore, the force Fi does not cause damage on the electronic circuits and the like, including the plurality of electronic components 15a, on the sensor substrate 15.

In addition, the output signals of the pressure sensors 14 are transmitted to the processor 4, subjected to the predetermined signal processing in the processor 4, and transmitted to the monitor 8. This allows the information acquired based on the output signals of the pressure sensors 14 to be presented to the operator or the like through the display on the monitor 8. Thus, the operator or the like can perform the insertion operation of the insertion portion 5 appropriately, while viewing the information displayed on the monitor 8. Accordingly, the present embodiment can contribute to the facilitation of the insertion operation of the insertion portion 5 and the improvement of the operability thereof.

In addition, in endoscope systems in recent years, an automatic insertion apparatus and the like of the insertion portion have been proposed and put into practical use. Therefore, if the endoscope 2 in the present embodiment is applied to the endoscope system including the automatic insertion apparatus of this type, the information acquired based on the output signals of the pressure sensors 14 can be utilized for automatic insertion/extraction control of the insertion portion. Accordingly, the present embodiment can contribute to the facilitation of the automatic insertion/extraction operation of the insertion portion 5 and the improvement of the operability thereof. For example, the processor 4 may be configured to automatically control at least one of an insertion direction or an insertion force of the insertion portion 5 based on output signals from the pressure sensors 14.

The above-described first embodiment shows the configuration example in which the load detection unit 22 is provided in the distal-end constituting portion 5a of the insertion portion 5 in the endoscope 2. However, the configuration of the present disclosure is not limited to the configuration example in the above-described first embodiment.

Next, a second embodiment of the present disclosure will be described below. The second embodiment of the present disclosure relates to an endoscope cap configured separately from the insertion portion 5, and configured to be attachable to and detachable from the distal end part of the insertion portion 5. In addition, the endoscope cap is configured by including a load detection unit such as pressure sensors.

The basic configuration of the load detection unit applied to the present embodiment is substantially the same as that of the load detection unit applied to the endoscope in the above-described first embodiment. The present embodiment shows a configuration example of the endoscope cap configured by including the respective constituent elements of the load detection unit (included in the distal-end constituting portion) in the endoscope of the above-described first embodiment. Therefore, in the present embodiment, the same components as those in the above-described first embodiment are attached with the same reference signs, detailed descriptions thereof will be omitted, and only the different configurations will be described.

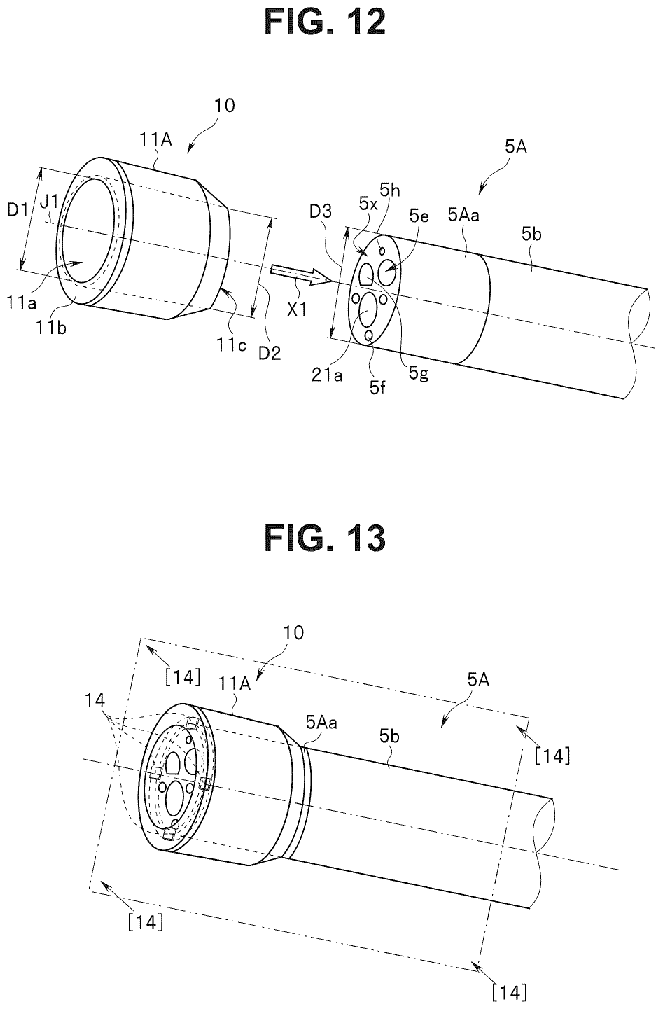

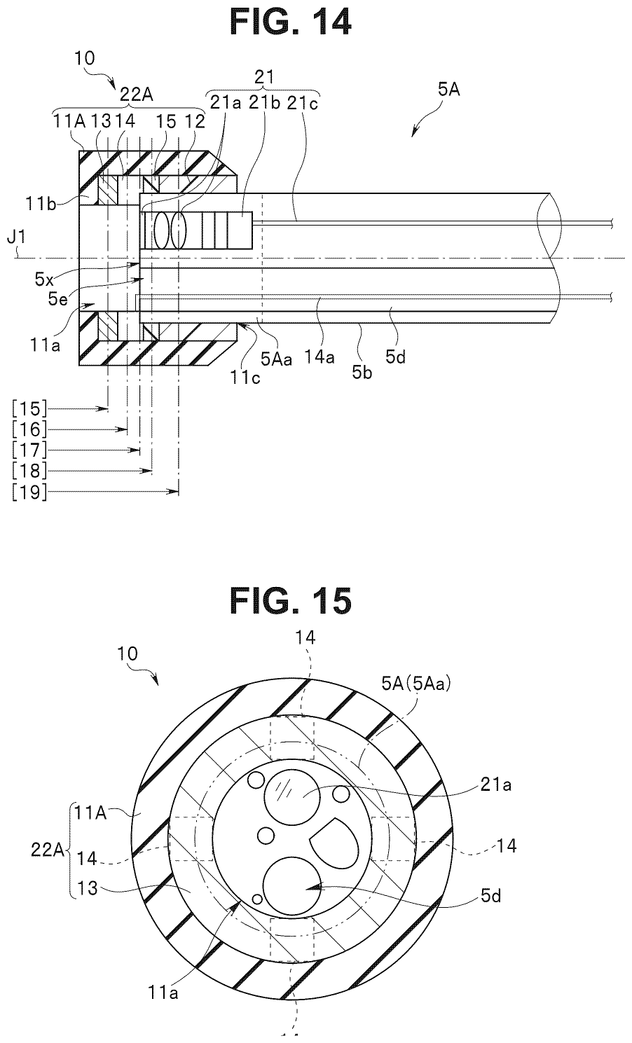

FIGS. 12 to 19 show the endoscope cap in the second embodiment of the present disclosure. Among these, FIGS. 12 to 14 each show, in an enlarged manner, the endoscope cap of the present embodiment and the distal end part of the insertion portion to which the endoscope cap is mounted. FIG. 12 is an exploded perspective view showing how the endoscope cap is mounted to the distal end part of the insertion portion. FIG. 13 is an appearance perspective view showing the state where the endoscope cap of the present embodiment is mounted to the distal end part of the insertion portion. FIG. 14 is a longitudinal sectional view along the imaginary plane indicated by the arrow [14] in FIG. 13.

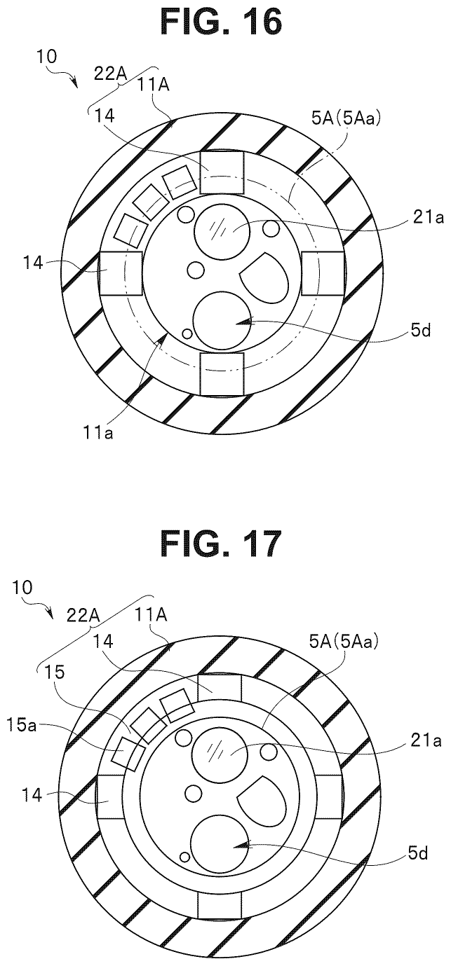

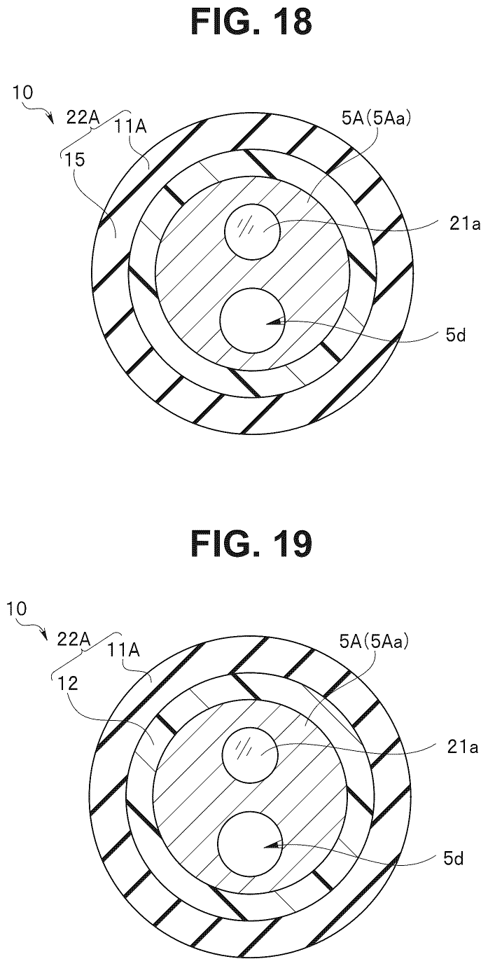

FIGS. 15 to 19 are enlarged cross-sectional views of the distal end part of the insertion portion, with the endoscope cap of the present embodiment being mounted. Among these, FIG. 15 is a cross-sectional view along the line indicated by the arrow [15] in FIG. 14. FIG. 16 is a cross-sectional view along the line indicated by the arrow [16] in FIG. 14. FIG. 17 is a cross-sectional view along the line indicated by the arrow [17] in FIG. 14. FIG. 18 is a cross-sectional view along the line indicated by the arrow [18] in FIG. 14. FIG. 19 is a cross-sectional view along the line indicated by the arrow [19] in FIG. 14.

Note that, in the cross-sectional views in FIGS. 15 to 19, only the constituent members included in the load detection unit are mainly shown, and illustrations of other constituent members are partly omitted in order to avoid the complication of the drawings.

An endoscope cap 10 of the present embodiment is a constituent member that is mounted to a distal-end constituting portion of an insertion portion in an endoscope included in an endoscope system of a general type, so as to cover a part (a part of the lateral surface and a part of the front surface) of the outside surface of the distal-end constituting portion. As shown in FIG. 13, when the endoscope cap 10 is mounted to a predetermined position of an insertion portion 5A of an endoscope, the endoscope cap 10 functions as a load detection device for detecting an amount of force (load) applied to the distal end part of the insertion portion 5A. In addition, the endoscope cap 10 is configured to be attachable to and detachable from a distal-end constituting portion 5Aa, as shown in FIGS. 12 to 14.

As described above, the endoscope cap 10 of the present embodiment is used in the state being mounted to the distal-end constituting portion of the insertion portion of the endoscope in the conventional general endoscope system. Therefore, the distal-end constituting portion 5Aa has an inner configuration substantially the same as that of the distal-end constituting portion applied to the conventional general endoscope. In view of the above, detailed description on the inner configuration of the distal-end constituting portion 5Aa will be omitted, and description will be briefly made below.

As shown in FIGS. 12 to 14, etc., the distal-end constituting portion 5Aa of the insertion portion 5A in the endoscope to which the endoscope cap 10 in the present embodiment is applied is of a general type including an image pickup unit 21 (see FIG. 14), an illumination unit (not shown), a treatment instrument insertion channel 5d (a part thereof), and the like. The configurations of the image pickup unit 21, the illumination unit, and the treatment instrument insertion channel 5d are substantially the same as those applied to the conventional endoscope of the same type.

For example, the image pickup unit 21 is a constituent unit including an image pickup lens 21a, an image pickup device 21b, a signal transmission cable 21c, and the like and configured to acquire a desired image (see FIG. 14). The illumination unit is a constituent unit including an illumination lens and configured to illuminate a target. The treatment instrument insertion channel 5d is a hollow tubular member including, at the distal end thereof, a channel opening 5e and configured to pass through the inside of the insertion portion 5A to reach the inside of the operation portion.

In addition, as shown in FIG. 12, etc., various constituent members such as the image pickup lens 21a as a part of the image pickup unit 21, an illumination lens 5f as a part of the illumination unit, a gas/liquid feeding nozzle 5g, an auxiliary liquid feeding port 5h, and the channel opening 5e are disposed on a front surface 5x of the distal-end constituting portion 5Aa, for example. Note that the distal-end constituting portion 5Aa in the present embodiment does not include the load detection unit.

The endoscope cap 10 in the second embodiment of the present disclosure is mounted to the distal-end constituting portion 5Aa thus configured. In other words, as shown in FIG. 12, etc., the endoscope cap 10 is mounted from a direction indicated by an arrow X1, which is along a center axis J1 of the insertion portion 5A, toward the distal end part of the distal-end constituting portion 5Aa. Thus, as shown in FIG. 13 and FIG. 14, the endoscope cap 10 is mounted so as to cover the lateral surface on the side closer to the distal end and a part of the front surface 5x, of the outside surface of the distal-end constituting portion 5Aa. Note that, in this case, the endoscope cap 10 is attachable to and detachable from the distal-end constituting portion 5Aa.

To this end, the endoscope cap 10 is formed in substantially a cylindrical shape, as a whole. The endoscope cap 10 includes, at one end portion (distal end side) thereof, a first opening 11a. The endoscope cap 10 includes, at another end portion (proximal end side) thereof, a second opening 11c. The first opening 11a and the second opening 11c are connected to each other by a through hole that penetrates the endoscope cap 10. Note that the first opening 11a is an opening formed in an inward flange 11b of a distal end cap 11A to be described later.

Here, as shown in FIG. 12, an inner diameter dimension D2 of the second opening 11c is formed to be substantially the same as or slightly larger than an outer diameter dimension D3 of the distal-end constituting portion 5Aa (D2≥D3). In addition, an inner diameter dimension D1 of the first opening 11a is formed to be smaller than the outer diameter dimension D3 of the distal-end constituting portion 5Aa (D3>D1).

Furthermore, the first opening 11a has a size (area) that allows exposure, without shielding, of all the various constituent members (the image pickup lens 21a, the illumination lens 5f, the gas/liquid feeding nozzle 5g, the auxiliary liquid feeding port 5h, the channel opening 5e, etc.) that are disposed on the front surface 5x of the distal-end constituting portion 5Aa, when the endoscope cap 10 is mounted to a predetermined position of the distal-end constituting portion 5Aa.

The endoscope cap 10 in the present embodiment includes a load detection unit 22A. The load detection unit 22A is a constituent unit disposed at the distal end part of the distal-end constituting portion 5Aa and configured to detect an amount of force (load) applied to the distal end part of the insertion portion 5A.

To this end, as shown in FIG. 14, etc., the endoscope cap 10 includes the distal end cap 11A, a proximal end cap 12, a rigid ring 13, pressure sensors 14, a sensor substrate 15, and the like.

The distal end cap 11A is an exterior member that mainly covers the lateral surface on the side closer to the distal end and a part (outer peripheral edge portion) of the front surface of the outside surface of the distal-end constituting portion 5Aa. The distal end cap 11A is formed in substantially a cylindrical shape as a whole. The distal end cap 11A includes, at one end portion (distal end side) thereof, the inward flange 11b in which the first opening 11a is formed. The distal end cap 11A includes, at another end portion (proximal end side) thereof, the second opening 11c. The first opening 11a and the second opening 11c are connected to each other by the through hole that penetrates the distal end cap 11A, as described above. Note that the distal end cap 11A is formed of a flexible material having low rigidity (for example, rubber material, etc.).

The distal end cap 11A includes, in the inner region thereof, the rigid ring 13, the pressure sensors 14, the sensor substrate 15, and the proximal end cap 12 that are disposed so as to align in this order from the distal end side toward the proximal end side.

The proximal end cap 12 is formed in substantially a cylindrical shape as a whole and inserted into the other end portion (proximal end side) of the distal end cap 11A. The proximal end cap 12 is formed by using a material (for example, resin material) whose rigidity is higher than that of the distal end cap 11A.

When the endoscope cap 10 is mounted to the distal-end constituting portion 5Aa (see FIG. 13 and FIG. 14), the distal-end constituting portion 5Aa is held in a state of being inserted into the proximal end cap 12. Therefore, the inner diameter of the proximal end cap 12 is set to be substantially the same as or slightly larger than the inner diameter of the second opening 11c of the endoscope cap 10. The rigid ring 13 is disposed, with the front end surface thereof being in contact with the inner surface of the inward flange 11b of the distal end cap 11A. In this case, the front end surface of the rigid ring 13 is fixed integrally with the inner surface of the inward flange 11b by adhering, for example. With such a configuration, the rigid ring 13 is disposed at a part which is closer to the distal end of the distal-end constituting portion 5Aa and which is in the vicinity of the outer peripheral edge portion of the distal-end constituting portion 5Aa.

The rigid ring 13 is formed by using a hard material (for example, a metal material such as stainless steel (e.g., SUS)) having high rigidity. Thus, the rigidity of the rigid ring 13 is higher than that of the distal end cap 11A.

The plurality of pressure sensors 14 are mounted on the mounting surface of the sensor substrate 15. A pressure-sensitive portion (front surface) of each of the pressure sensors 14 is arranged so as to face the rear end surface of the rigid ring 13. At this time, the pressure-sensitive portion of each of the pressure sensors 14 and the rear end surface of the rigid ring 13 are arranged facing each other, with almost no gap therebetween or with a slight gap (for example, approximately 0.1 mm) therebetween.

The plurality of pressure sensors 14 are arranged at substantially equal intervals along the circumferential direction around the center axis J1 of the distal end cap 11A. At least three pressure sensors 14 may be provided. Note that, for example, piezoelectric elements are used as the pressure sensors 14.

The sensor substrate 15 has substantially a circular ring shape, and is disposed such that the outer circumferential surface thereof is along the inner wall surface of the lateral surface of the distal end cap 11A. The plurality of pressure sensors 14 are mounted on the mounting surface (front surface side) of the sensor substrate 15. In addition, on the same mounting surface of the sensor substrate 15, an electronic circuit (for example, an AD conversion circuit) that receives the output signals of the pressure sensors 14 to perform predetermined signal processing on the received signals is mounted. In addition, the rear surface side of the sensor substrate 15 is secured to the distal end surface of the proximal end cap 12.

Note that, in order to avoid complication of the drawings, illustration of the electronic circuit such as the AD conversion circuit is partly omitted. Although detailed description will be made later, FIG. 16, FIG. 17, etc., each partly show, in a conceptual manner, the state where the plurality of electronic components 15a as the members constituting the AD conversion circuit, etc., are mounted on the mounting surface of the sensor substrate 15.

As shown in FIG. 14, a signal cable 14a extends from the pressure sensors 14 (the sensor substrate 15 on which the pressure sensors 14 are mounted) toward the proximal end side of the insertion portion 5A. The signal cable 14a passes through the inside of the treatment instrument insertion channel 5d of the insertion portion 5A. In addition, although illustration is omitted, the signal cable 14a is extended, for example, from a treatment instrument insertion port 6d, and then connected to the processor 4 through a predetermined connector (not shown). Such a configuration allows the output signals of the pressure sensors 14 to be transmitted to the processor 4.

The endoscope cap 10 thus configured functions as a detection device that detects an amount of force (load) applied mainly to the front surface 5x of the distal end part of the insertion portion 5A, when the endoscope cap 10 is mounted to the distal-end constituting portion 5Aa of the insertion portion 5A.

The operation of the endoscope cap 10 in the present embodiment thus configured is substantially the same as that in the above-described first embodiment. Note that the present embodiment is different in that an endoscopy or the like is performed by the endoscope cap 10 being mounted to the distal-end constituting portion 5Aa of the insertion portion 5A of the endoscope, according to a predetermined procedure. Note that when the endoscope cap 10 is mounted to the distal-end constituting portion 5Aa of the insertion portion 5A of the endoscope, the signal cable 14a is inserted into the treatment instrument insertion channel 5d.

The second embodiment thus configured is also capable of attaining substantially the same effects as those of the above-described first embodiment.

Furthermore, according to the present embodiment, the number of wirings of the signal cable 14a for transmitting the output signals of the pressure sensors 14 can be reduced. Therefore, the signal cable 14a can be arranged by being inserted through the treatment instrument insertion channel 5d, for example.

Such a configuration can avoid the configuration in which the wirings, etc., are extended along the outer lateral surface of the insertion portion when the endoscope cap 10 is mounted to the insertion portion 5A, which contributes to an improvement of the operability in the insertion operation of the insertion portion 5A.

The present disclosure is not limited to the above-described embodiments, and it goes without saying that various modifications and applications can be implemented within a range without departing from the gist of the disclosure. Furthermore, the above embodiments include disclosures at various stages, and various disclosures can be extracted by appropriate combinations of a plurality of disclosed constituent elements. For example, even if some of the constituent elements are removed from all the constituent elements shown in the above-described one embodiment, a configuration from which the constituent elements are removed can be extracted as a disclosure insofar as the configuration can solve the problem to be solved by the disclosure and attain the effects of the disclosure. Furthermore, constituent elements over different embodiments may be appropriately combined. The disclosure is not limited by the specific embodiments except as limited by appended claims.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:
Clause 1: an endoscope comprising:
   an insertion portion; and
   pressure sensors including pressure-sensitive portions, respectively, wherein
   the pressure sensors are arranged at a part closer to a distal end inside the insertion portion, and
   the insertion portion includes a first region that covers the pressure-sensitive portions and a second region including an outer surface of the insertion portion, and rigidity of the first region is higher than rigidity of the second region.
Clause 2: The endoscope according to clause 1, wherein
   a contact member is further provided at a position with which the pressure-sensitive portions of the pressure sensors are in contact, and rigidity of the contact member is higher than the rigidity of the second region, and
   a force applied to the outer surface is transmitted to the pressure-sensitive portions of the pressure sensors through the contact member.
Clause 3: The endoscope according to clause 2, wherein
   the pressure sensors include at least two pressure sensors that are provided along a circumferential direction of a distal end of the insertion portion,
   the first region includes an inner contact surface with which the respective pressure-sensitive portions of the pressure sensors are in contact, and
   the contact member is arranged between the second region and the pressure-sensitive portions, and configured to transmit the force applied to the outer surface to the at least two pressure sensors.
Clause 4: The endoscope according to clause 3, wherein
   the contact member is arranged across the respective pressure-sensitive portions of the at least two pressure sensors, and
   a gap is provided between a surface coplanar with the pressure-sensitive portions and a surface on which the pressure sensors are mounted.
Clause 5: The endoscope according to clause 1, wherein the pressure sensors include at least three pressure sensors arranged at equal intervals along a circumferential direction of the insertion portion.
Clause 6: The endoscope according to clause 3, wherein
   an analog-to-digital (AD) conversion section is further provided between the at least two pressure sensors in a circumferential direction, and
   the AD conversion section is configured to control signals of the pressure sensors.
Clause 7: The endoscope according to clause 2, wherein the contact member is a member formed in a circular ring shape, and having a thickness of 0.005 mm to 3 mm and a diameter of 5 mm to 20 mm.
Clause 8: The endoscope according to clause 2, wherein the contact member is formed of a metal or a hard resin.
Clause 9: The endoscope according to clause 1, further comprising:
   a control apparatus; and
   a display apparatus, wherein
   the control apparatus is configured to process output signals from the pressure sensors, and acquire information on a direction of a force detected by the pressure sensors, and
   the display apparatus is configured to display the information on the direction of the force acquired by the control apparatus.
Clause 10: An endoscope cap that is attached to a distal end of an insertion portion of an endoscope, the endoscope cap comprising
   pressure sensors provided inside the endoscope cap, the pressure sensors including pressure-sensitive portions, respectively, wherein
   rigidity of a first region that covers the pressure-sensitive portions is higher than rigidity of a second region including an outer surface of the insertion portion.
Clause 11: The endoscope cap according to clause 10, wherein
   the first region includes an inner contact surface with which the respective pressure-sensitive portions of the pressure sensors are in contact,
   a contact member is further provided, and has the inner contact surface that is positioned in contact with the pressure-sensitive portions, and
   a force applied to the outer surface is transmitted to the pressure-sensitive portions through the contact member.
Clause 12: The endoscope cap according to clause 11, wherein
   the pressure sensors include at least two pressure sensors,
   the contact member is arranged between the second region and the pressure-sensitive portions, and
   the force applied to the outer surface is transmitted to the at least two pressure sensors.
Clause 13: The endoscope cap according to clause 12, wherein
   the contact member is arranged across the respective pressure-sensitive portions of the at least two pressure sensors, and
   a gap is provided between a surface coplanar with the pressure-sensitive portions and a surface on which the pressure sensors are mounted.
Clause 14: The endoscope cap according to clause 11, wherein
   the pressure sensors include wiring for transmitting signals, and
   the wiring is passed through a treatment instrument insertion channel of the endoscope.
Clause 15: The endoscope cap according to clause 10, wherein the pressure sensors include at least three pressure sensors arranged at equal intervals along a circumferential direction of the insertion portion.
Clause 16: The endoscope cap according to clause 12, wherein
   an analog-to-digital (AD) conversion section is further provided between the at least two pressure sensors in a circumferential direction, and
   the AD conversion section controls signals of the pressure sensors.
Clause 17: The endoscope cap according to clause 11, wherein the contact member is a member formed in a circular ring shape, and having a thickness of 0.005 mm to 3 mm and a diameter of 5 mm to 20 mm.
Clause 18: The endoscope cap according to clause 11, wherein the contact member is formed of a metal or a hard resin.

What is claimed is:

1. An endoscope comprising:
pressure sensors including respective pressure-sensitive portions; and
an insertion portion comprising a distal-end region where the pressure sensors are arranged inside the distal-end region, the distal-end region including:
a first region covering the pressure-sensitive portions and having a first rigidity higher than a second rigidity; and
a second region including an outer surface of the insertion portion and having the second rigidity.

2. The endoscope according to claim 1, further comprising a contact member positioned substantially in contact with the pressure-sensitive portions of the pressure sensors, the contact member having a rigidity higher than the second rigidity,
wherein a force applied to the outer surface of the insertion portion is transmitted to the pressure-sensitive portions through the contact member.

3. The endoscope according to claim 2, wherein
the pressure sensors include at least two pressure sensors arranged circumferentially within the distal-end region,
the first region includes the contact member, the contact member having a contact surface substantially in contact with the respective pressure-sensitive portions of the pressure sensors, and
the contact member is disposed between the second region and the pressure-sensitive portions and is configured to transmit the force applied to the outer surface of the insertion portion to the at least two pressure sensors.

4. The endoscope according to claim 3, wherein
the contact member is disposed across the respective pressure-sensitive portions of the at least two pressure sensors, and
a gap is provided between a reference surface coplanar with the pressure-sensitive portions and a mounting surface on which the pressure sensors are mounted.

5. The endoscope according to claim 1, wherein the pressure sensors include at least three pressure sensors arranged at equal intervals circumferentially within the distal-end region.

6. The endoscope according to claim 3, further comprising an analog-to-digital (AD) converter disposed between the at least two pressure sensors in a circumferential direction of the distal-end region and configured to perform analog-to-digital conversion on signals from the pressure sensors.

7. The endoscope according to claim 2, wherein the contact member is formed in a circular ring shape and has a thickness of 0.005 mm to 3 mm and a diameter of 5 mm to 20 mm.

8. The endoscope according to claim 2, wherein the contact member includes at least one of a metal or a hard resin.

9. The endoscope according to claim 1, further comprising:
a processor configured to process output signals from the pressure sensors and to acquire information on a direction of a force detected by the pressure sensors; and
a display configured to display the information on the direction of the force acquired by the processor.

10. An endoscope cap configured to be detachably attached to a distal end of an insertion portion of an endoscope, the endoscope cap comprising
pressure sensors disposed inside the endoscope cap and including respective pressure-sensitive portions;
a first region covering the pressure-sensitive portions and having a first rigidity higher than a second rigidity; and
a second region including an outer surface of the endoscope cap and having the second rigidity.

11. The endoscope cap according to claim 10, wherein
the first region includes a contact member, the contact member having a contact surface substantially in contact with the respective pressure-sensitive portions of the pressure sensors, and
a force applied to the outer surface of the endoscope cap is transmitted to the pressure-sensitive portions through the contact member.

12. The endoscope cap according to claim 11, wherein
the pressure sensors include at least two pressure sensors,
the contact member is disposed between the second region and the pressure-sensitive portions, and
the force applied to the outer surface of the endoscope cap is transmitted to the at least two pressure sensors.

13. The endoscope cap according to claim 12, wherein
the contact member is disposed across the respective pressure-sensitive portions of the at least two pressure sensors, and
a gap is provided between a reference surface coplanar with the pressure-sensitive portions and a mounting surface on which the pressure sensors are mounted.

14. The endoscope cap according to claim 11, wherein
the pressure sensors are connected to a signal cable configured to transmit signals from the pressure sensors, and
the signal cable is routed through a treatment instrument insertion channel of the endoscope.

15. The endoscope cap according to claim 10, wherein the pressure sensors include at least three pressure sensors arranged at equal intervals circumferentially within the endoscope cap.

16. The endoscope cap according to claim 12, further comprising an analog-to-digital (AD) converter disposed between the at least two pressure sensors in a circumferential direction of the endoscope cap and configured to perform analog-to-digital conversion on signals from the pressure sensors.

17. The endoscope cap according to claim 11, wherein the contact member is formed in a circular ring shape and has a thickness of 0.005 mm to 3 mm and a diameter of 5 mm to 20 mm.

18. The endoscope cap according to claim 11, wherein the contact member includes at least one of a metal or a hard resin.

19. An endoscope system comprising:
the endoscope according to claim 1; and
a processor configured to automatically control at least one of an insertion direction or an insertion force of the insertion portion based on output signals from the pressure sensors.

20. The endoscope system according to claim 19, further comprising a display, wherein
the processor is further configured to compute a distribution of a force applied to a distal end of the insertion portion based on the output signals from the pressure sensors, and
the display is configured to visualize the computed force distribution.
